(19) United States
(12) Patent Application Publication
(10) Pub. No.: US 2018/0235941 A1
(43) Pub. Date: Aug. 23, 2018

(54) PEDIATRIC ORAL SUSPENSION FORMULATIONS OF AMOXICILLIN AND CLAVULANATE POTASSIUM AND METHODS FOR USING SAME

(71) Applicant: UNIVERSITY OF PITTSBURGH-OF THE COMMONWEALTH SYSTEM OF HIGHER EDUCATION, Pittsburgh, PA (US)

(72) Inventor: Alejandro Hoberman, Wexford, PA (US)

(21) Appl. No.: 15/945,515

(22) Filed: Apr. 4, 2018

Related U.S. Application Data
(63) Continuation-in-part of application No. 14/371,731, filed on Jul. 10, 2014, now Pat. No. 9,987,257.
(60) Provisional application No. 62/481,381, filed on Apr. 4, 2017.
(60) Provisional application No. 61/585,234, filed on Jan. 10, 2012.

(51) Int. Cl. A61K 31/43; A61K 31/424; A61K 45/06; A61P 31/00; A61P 27/16; A61P 31/04

(52) U.S. Cl. CPC A61K 31/43; A61K 31/424; A61P 31/04; A61P 31/00; A61P 27/16; A61K 45/06

(57) ABSTRACT

The invention is directed to a pediatric oral suspension composition containing amoxicillin and clavulanate potassium where the clavulanate potassium is present in an amount equal to or less than about 21.5 mg/5 mL, and a method of treating bacterial infections by providing between about one to about fourteen dosage days of the composition. Also disclosed are methods of treating a patient comprising administering amoxicillin and clavulanate potassium to the patient in dosages of at least about 80 mg/kg/day of the amoxicillin and from about 1.66 mg/kg/day to about 2.84 mg/kg/day of the clavulanate potassium. The methods are useful for treating pediatric otitis media, treating a drug resistant bacterial infection, or treating beta-lactamase producing Haemophilus influenzae or Moraxella catarrhalis in a patient under 24 months of age.

### RELATED APPLICATIONS

This application claims priority from provisional application Ser. No. 62/481,381 filed Apr. 4, 2017, and is a continuation-in part of U.S. nonprovisional application Ser. No. 14/371,731, filed Jul. 10, 2014, which claims priority to provisional application Ser. No. 61/585,234 filed Jan. 10, 2012, all of which are incorporated by reference herein in their entireties.

### FIELD OF THE INVENTION

The disclosure generally relates to use of pediatric oral suspensions containing amoxicillin and clavulanate potassium in treating otitis media and other bacterial infections.

### BACKGROUND OF THE INVENTION

For pediatric administration of supplements and pharmaceuticals it is well recognized by those of skill in the art that solutions or liquid suspensions are highly preferable dosage forms. Tablets and capsules are difficult for children to swallow and the amount of drug delivered is not as flexible as is often required for pediatric drugs. With liquid dosage forms, by contrast, the amount of drug delivered to the patient can be varied over a wide range merely by regulating the volume of dose of known concentrations.

From the perspectives of ease of use, accuracy of dose, and bioavailability, oral liquid dosage forms are generally preferred to be in the form of a solution. From the perspective of taste, oral liquid dosage forms are generally preferred to be in the form of a suspension which tends to mask the taste of the drug. This is essentially useful with pediatric treatments as children generally do not like the taste of medicines. If the taste is not pleasing, the child can spit it out and therefore affect the treatment regimen. Especially for pediatric use, where doses are relatively small, accuracy and precision of dose is extremely important. For this reason, the preferable oral liquid form for many antibiotics for children is an oral suspension.

Amoxicillin is well known as a treatment for various bacterial infections and its use as an antibiotic, alone or in combination with other compositions and medications has been documented. However, treatment of certain bacterial infections has been made more difficult by resistance. In particular, many Gram-negative bacteria produce an enzyme, β-lactamase, that attacks the β-lactam ring of β-lactam antibiotics and renders them ineffective. To counteract this effect, β-lactamase inhibitors have been developed that can bind to β-lactamase and prevent it from attacking the antibiotic. The antibiotic and the inhibitor are preferably administered together. For example, the β-lactam antibiotic amoxicillin can be administered with the β-lactamase inhibitor clavulanate potassium. This additional clavulanate potassium is not needed in non-beta lactamase mediated resistance treatments.

Moreover, the amount of amoxicillin has increased in dosage as certain bacteria have become resistant to the amoxicillin. For instance, Streptococcus pneumoniae have become resistant to amoxicillin such that the prescribed treatment dosage has increased from 400 mg/5 mL per day to over 600 mg/5 mL per day over the last decade. The amount of the clavulanate potassium has similarly increased or remained constant and in ratio with the increased amount of amoxicillin per dosage. Further the actual combined taken dosage for the patient, of the combined amoxicillin and clavulanate potassium has in fact doubled over the last few years, such as taking the dosage 2 or 3 times per day during treatment.

A combination of amoxicillin and clavulanate potassium is a treatment of choice for acute otitis media. Symptoms of acute otitis media include fever, otalgia, irritability and/or pain, fussiness, tugging or rubbing or holding of the ears, sleeping and feeding disturbances, and infrequently, perforation of the tympanic membrane. Most of these symptoms are mild to moderate and will eventually resolve spontaneously. The combination of amoxicillin and clavulanate potassium is considered the gold standard for antibiotic treatment, against which most new products on the market are compared.

Acute otitis media remains the most frequently occurring infection for which antimicrobial agents are prescribed for children in the United States. Concerns about the development of antimicrobial resistance have led to recommendations to withhold antibiotics from such children unless symptoms persist or worsen, which is sometimes referred to as a “watchful waiting strategy”, which can prolong the acute otitis media symptoms for the child.

Notably, due to vaccination there has been a selective reduction of treatable S. pneumoniae compared to resistant Haemophilus influenzae as causative agents. The resistance building up to amoxicillin has led to the increase of the dosage and/or dosage unit of amoxicillin in certain antibiotic compositions. Correspondingly, the other active pharmaceutical ingredients in such antibiotic compositions has also increased, typically based on ratios. Further, in addition to H. influenzae, another beta-lactamase producing bacteria of Moraxella catarrhalis is also seen in otitis media, although to a much lesser extent than H. influenzae.

Formulations of amoxicillin and clavulanate potassium have used varying ratios of the two components; over time, the trend has been to increase the dosage of amoxicillin, mainly to achieve higher efficacy rates against S. pneumoniae. Amoxicillin-clavulanate potassium ratios have thus ranged from 4:1 to 14:1. The currently available commercial amoxicillin-clavulanate potassium suspension for pediatric use contains 600 mg of amoxicillin and 42.9 mg of clavulanate potassium per 5 mL (a ratio of 14:1). The currently recommended pediatric dosage, 90/6.4 mg/kg/day administered in two divided doses for 10 days, results in a dose of clavulanate potassium almost twice as high as the dose recommended for adults (6.4 mg/kg/day vs. 3.5 mg/kg/day). The currently recommended adult dosage ranges from 500 mg/250 mg-4,000 mg/250 mg per day with a common dose being 1700 mg/250 mg per day.

Certain current formulations of amoxicillin and clavulanate potassium have a high concentration of clavulanate potassium. However, clavulanate potassium has the potential to cause rare serious side effects such as jaundice and hepatitis (see, for example, Joint Formulary Committee. British National Formulary, 47th edition. London: British Medical Association and Royal Pharmaceutical Society of Great Britain; 2004). Other minor systemic reactions include headache, rash, mycosis, vaginitis, and agitation. The following infrequent and rare adverse reactions have been reported for ampicillin-class antibiotics: hepatitis; cholestatic jaundice; hemorrhagic/pseudomembranous colitis; angioedema; Stevens-Johnson syndrome; hypersensitivity vasculitis; tooth discoloration; and seizure.

More frequently, in pediatric patients clavulanate potassium can cause diarrhea (Reed, M. D. (1998), Pediatric Infectious Disease Journal, 17, 957-62), which can lead to dehydration in such young patients and further sickness. Although advantageous from the standpoint of efficacy, use of amoxicillin and clavulanate potassium is also associated with a relatively high incidence of diarrhea. This diarrhea is infrequently severe enough to require discontinuing treatment, but it can cause delays in children's returning to day care and in parents' returning to work. While not being bound by theory, it is possible that the occurrence of diarrhea is related to the clavulanate potassium component of the drug combination.

Clavulanate potassium can also cause vomiting and diaper rash in children. These more common side effects of diarrhea, diaper rash, vomiting and oral moniliasis, while not as serious as the other side effects, are debilitating to the care givers of the pediatric patient. The pediatric patient with diarrhea and/or vomiting cannot return to school or day care until typically twenty-four (24) hours after the last episode of diarrhea or vomiting. Such constraints affect the parents and care giver of the pediatric patient in that they typically must use vacation days to stay home with the vomiting child, or work from home with a reduced productive outcome. Given the data that approximately twenty percent (20%) of all pediatric patients taking the current dosage of amoxicillin and clavulanate potassium experience some diarrhea and/or vomiting, this translates to twenty percent (20%) of children not able to return to school or day care and consequently twenty percent (20%) of parents or care givers staying home with the affected pediatric patient. It is thus discovered and sought to minimize the amount of clavulanate potassium necessary for treatment of children to be very useful.

While not wishing to be bound by theory, it is possible that clavulanate potassium has the property of binding irreversibly to β-lactamase. (Reed, M. D. (1998), Pediatric Infectious Disease Journal, 17, 957-62), thus enhancing the effectiveness of amoxicillin. If so, then this might explain the rapidly declining need for clavulanate potassium in the course of a combined treatment with amoxicillin and clavulanate potassium. After an initial loading dose of clavulanate potassium is provided, either the same amount or much less can be needed. Continuing to administer the same composition of amoxicillin and clavulanate potassium over the treatment regimen period, as the currently prescribed method, can result in too much clavulanate potassium being taken in subsequent doses.

Clearly for a pediatric population, it is particularly important to use the minimal, yet effective, amount of clavulanate potassium so as to reduce the risk of diarrhea, diaper dermatitis and vomiting in a young population. Disclosed herein are therapeutic methods which use less clavulanate potassium over the entire treatment regimen, which can either be (a) a lower dose through the regimen, (b) high dose of clavulanate potassium at the beginning of treatment to bind to β-lactamase and then less throughout the regimen, or (c) a combination thereof and varying dosages throughout the treatment regimen. Thus, either the same smaller amount, or less, clavulanate potassium can be required as treatment progresses. However, in current treatments, amoxicillin and clavulanate potassium are administered in a set combined dosage form administered at the same level at a ratio of about 4:1-14:1 over a period of days (typically ten (10) days). Although various amoxicillin and clavulanate potassium regimens are available, the need for minimizing clavulanate potassium has not been adequately addressed. Until recently, the thrust behind reformulations of amoxicillin-clavulanate potassium has been adequate coverage of S. pneumoniae. Currently, focus has shifted to adequate coverage of H. influenzae. However, minimizing adverse events and side effects has not been given precedence over the efficacy of the dosage formulation to address S. pneumoniae, H. influenzae and other causes of infections. For instance, a study published in the New England Journal of Medicine in January 2011 showed higher rates of diarrhea, vomiting and diaper dermatitis in children taking amoxicillin-clavulanate potassium versus those taking a placebo. See Hoberman et al., New Engl. J. Med., 2011; 364:105-15.

Thus, a need exists for an amoxicillin-clavulanate potassium composition, and treatment method, which reduces the side effects of diarrhea, vomiting and diaper dermatitis in children, while still maintaining the high efficacy of the antibiotic combination. One of the objectives of the invention is to maintain high efficacy while improving safety profile, namely reducing the common and disruptive side effects of diarrhea, vomiting and diaper dermatitis in pediatric patients.

Various formulations and dosing modalities currently exist for the combination of amoxicillin-clavulanate potassium. Tablets and suspensions are also available. Delayed release tablet formulations have been developed (see, for example, U.S. Pat. Nos. 5,910,322; 6,299,903; 6,544,558; 6,756,057; 6,783,773; 6,977,086; 7,122,204; 7,534,781; and publications 2006/0121106, 2008/0300569, and 2011/0020408, each of which are incorporated by reference herein).

However, these systems provide combination doses of amoxicillin-clavulanate potassium that do not address the need for a reduced set amount of clavulanate potassium (whether constant throughout the treatment or in a reduced set amount when compared to current conventional and known amounts) throughout the treatment regimen, including reducing the amount as treatment progresses. There is a need for a dosage, and a method that provides a means of reducing the overall amount of clavulanate potassium for the treatment regimen, when compared to current conventional and known dosage amounts and methods of treatment.

Thus, a need exists for a pediatric oral suspension composition having amoxicillin-clavulanate potassium to maintain the efficacy of the composition in view of beta-lactamase mediated resistance H. influenzae and M. catarrhalis, without elevating the possibility of the severe side effects of jaundice and hepatitis and the more common and disruptive side effects of diarrhea, diaper rash and vomiting.

These and other needs are met by the disclosed compositions and methods for treating bacterial infections, including acute otitis media and other respiratory infections such as, but not limited to, acute bacterial rhinosinusitis. Other advantages of the present disclosure will become apparent from the following description and appended claims.

### SUMMARY OF THE INVENTION

This invention provides an oral suspension composition for pediatric use including an amount of amoxicillin and an amount no greater than 21.5 mg/5 mL of clavulanate potassium.

Also part of this invention is a method of treatment for acute otitis media in pediatric patients including multiple days of dosage of a composition of amoxicillin and clavulanate potassium, with the clavulanate potassium present in an amount no greater than 21.5 mg/5 mL, and a method including multiple days of dosage, where the clavulanate potassium amount is constant or reduced over the dosage days. This invention includes methods for treating conditions such as acute otitis media in children.

Also part of this invention is an oral suspension composition for pediatric use including an amount of amoxicillin and an amount of clavulanate potassium in at a ratio of at least 26:1, preferably in a range of 28:1 through 56:1.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same element(s) throughout the figures.

FIG. 1 is a table showing selected demographic and clinical characteristics of children according to amoxicillin/clavulanate (A/C) treatment regimen. In the figure, the following notations are made: * denotes race and ethnicity were reported by the children's parents. † denotes comparison of Phase 1 children vs. historical controls, white vs. nonwhite; P=0.02; and comparison of Phase 2 children vs. historical controls, white vs. nonwhite; P<0.001. ‡ denotes comparison of Phase 2 children vs. historical controls, P=0.006. § denotes exposure to other children was defined as exposure to at least three children for at least 10 hours per week. ∥ denotes the Acute Otitis Media Severity of Symptoms (AOM-SOS) scale consists of five discrete items—tugging of ears, crying, irritability, difficulty sleeping, and fever. Parents are asked to rate these symptoms, in comparison with the child's usual state, as “none,” “a little,” or “a lot,” with corresponding scores of 0, 1, and 2. Thus, total scores range from 0 to 10, with higher scores indicating greater severity of symptoms. ¶ denotes comparison of Phase 2 children vs. historical controls, P=0.06. ** denotes comparison of Phase 2 children vs. historical controls, P=0.08.

FIG. 2 is a table showing adverse side-effects, clinical efficacy, and symptomatic response according to amoxicillin/clavulanate (A/C) treatment regimen*. In the figure, the following notations are made: * denotes data were missing for some children for some analyses. † denotes PDD refers to protocol-defined diarrhea. ‡ denotes comparison of historical controls vs. Phase 2 children; logistic regression. § denotes comparison of historical controls vs. Phase 2 children, log rank test. ∥ denotes comparison of historical controls vs. Phase 2 children, T-test. ¶ denotes study medication was discontinued only because of diaper dermatitis in 2 children. ** denotes comparison of historical controls vs. Phase 2 children, generalized estimated equations. †† denotes the Acute Otitis Media Severity of Symptoms (AOM-SOS) scale consists of five discrete items—tugging of ears, crying, irritability, difficulty sleeping, and fever. Parents are asked to rate these symptoms, in comparison with the child's usual state, as “none,” “a little,” or “a lot,” with corresponding scores of 0, 1, and 2. Thus, total scores range from 0 to 10, with higher scores indicating greater severity of symptoms. Restricted to children with AOM-SOS≥3 at enrollment. The cutoff between ≤50% and >50% was based on data from a study of minimal important difference in AOM-SOS scores.¹⁷ §§ denotes scores on the following scale: 1—very dissatisfied; 2—somewhat dissatisfied; 3—neutral; 4—somewhat satisfied; 5—very satisfied.

FIG. 3 is a table showing pharmacokinetic profile following administration of a single dose of standard, Phase 1, and Phase 2 amoxicillin-clavulanate (A/C) formulations, respectively. In the figure, the following notations are made: A/C denotes amoxicillin-clavulanate. Cmax denotes maximum serum concentration. Tmax denotes the time after administration when the maximum serum concentration was reached. AUC₀₋ₜ denotes area under the plasma concentration-time curve from the beginning to the end of the dosing interval (12 hours). The last actual sample was obtained 4 hours after administration of medication, and the AUC from 4 to 12 hours after administration was projected using the estimated terminal half-life. T1/2 denotes half-life. CL/F denotes apparent total clearance of the drug from plasma after oral administration. * denotes data taken from the Augmentin Prescribing Information. † denotes when SD and range are not provided, data were obtained from a single sample.

FIG. 4(A-D) are images of tympanic membranes. The images show tympanic membranes having a normal size (FIG. 4A), slightly bulging (FIG. 4B), moderate bulging (FIG. 4C), and marked bulging (FIG. 4D).

FIG. 5 is a graph showing percentages of children with protocol-defined diarrhea (PDD) according to day of treatment and amoxicillin/clavulanate dosage regimen.

FIG. 6 is a set of graphs showing population plasma concentration vs. time curves for children receiving the reduced-clavulanate formulation of amoxicillin-clavulanate at varying dosage regimens during Phase 1 (90/3.2 mg/kg/day) and Phase 2 (80/2.85 mg/kg/day) trials. Plasma concentrations for 6, 8, 10, 12 hours were extrapolated based on elimination rate constant calculated from data collected up to 4 hours. Data points up to 4 hours without a standard deviation bar indicate that the assessment was available for only one child.

### DETAILED DESCRIPTION OF THE INVENTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Unless defined otherwise below, all technical and scientific terms used herein have the same meaning as commonly understood to one of ordinary skill in the art to which this invention belongs.

### Terminology

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to the compound are discussed, specifically contemplated is each and every combination and permutation of the compound and the modifications that are possible unless specifically indicated to the contrary. Thus, if a class of compounds A, B, and C are disclosed as well as a class of compounds D, E, and F and an example of a combination compound, or, for example, a combination compound comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “can,” “may,” “optionally,” “can optionally,” and “may optionally” are used interchangeably and are meant to include cases in which the condition occurs as well as cases in which the condition does not occur. Thus, for example, the statement that a formulation “may include an excipient” encompasses cases in which the formulation includes an excipient as well as cases in which the formulation does not include an excipient.

“Unit Dose” means a single dose of a composition given once, in a single administration.

“Dose” or “dosage” can mean either a single administration of a composition or can mean two or more (e.g., several) administrations of the same composition depending on context. For example, if the composition is given twice a day, a dose could mean two administrations of the same composition, in suitably measured amounts. Thus, the same “dose” can be given two or three times (or more if necessary) in the treatment regimen before progressing to the subsequent dose, which would be of a composition having a different given amount of medication. However, as defined above, a unit dose means a single dose given a single time, i.e. in one administration.

“Dosage form” is the type of formulation in which the compositions of this invention are administered, such as but not limited to amoxicillin-clavulanate potassium. A dosage form can be a discrete unit such as a tablet or can be a liquid form or a suspension, from which unit dosages are measured.

“Patient” can be any living being that can be treated with a composition of this invention. The patient is preferably a human child, but can also be an adult or non-human such as an animal. The patient can be a male or female of any race, creed, ethnicity, socio-economic status, or other general classifiers. In some embodiments, the patient is under 24 months of age.

The terms “about” and “approximately” are defined as being “close to” as understood by one of ordinary skill in the art. In some non-limiting embodiments, the terms are defined to be within 10% of the associated value provided. In some non-limiting embodiments, the terms are defined to be within 5%. In still other non-limiting embodiments, the terms are defined to be within 1%.

Ranges can be expressed as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Amoxicillin” and “clavulanate potassium” or “clavulanic acid” refer to any existing forms of the compounds amoxicillin and clavulanate potassium such as acid and salt forms, whether alkali, alkaline, or acid salts, polymorphs, hydrates, solvates, racemates and mixtures. Examples are amoxicillin trihydrate or sodium, and potassium clavulanate. The weights of amoxicillin and clavulanate potassium refer to weight in equivalents of corresponding free acids unless otherwise indicated. The weights used in a formulation can also be adjusted by known methods depending on potency.

“Comprising” is intended to mean that the compositions, methods, etc. include the recited elements, but do not exclude others. It is expressly understood that where the compositions, systems, or methods use the term comprising, the specification also discloses the same compositions, systems, or methods using the terms “consisting essentially of” and “consisting of” as it relates to the modified elements.

Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

The terms “treat,” “treating,” “treatment,” and grammatical variations thereof as used herein, include partially or completely delaying, curing, healing, alleviating, relieving, altering, remedying, ameliorating, improving, stabilizing, mitigating, and/or reducing the intensity or frequency of one or more diseases or conditions, symptoms of a disease or condition, or underlying causes of a disease or condition. Treatments according to the invention may be applied prophylactically, palliatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms.

In some instances, the terms “treat”, “treating”, “treatment” and grammatical variations thereof, include eliminating or reducing the amount of bacteria present in an infection. In some instances, the terms “treat”, “treating”, “treatment” and grammatical variations thereof, include eliminating or reducing the growth or spreading of bacteria present in an infection. In some instances, the terms “treat”, “treating”, “treatment” and grammatical variations thereof, include eliminating or reducing discomfort and/or pain associated with a bacterial infection. Measurements of treatment can be compared with prior treatment(s) of the subject, inclusive of no treatment, or compared with the incidence of such symptom(s) in a general or study population.

### Amoxicillin and Clavulanate Compositions

Disclosed herein are compositions comprising amoxicillin and clavulanate potassium. Clavulanate potassium includes clavulanic acid, which is the generic name for (2R,5R,Z)-3-(2-hydroxyethylidene)-7-oxo-4-oxa-1-azabicyclo[3.2.0]heptane-2-carboxylic acid, which is a known compound of the following formula:

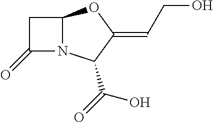 [SMILES: O=C(O)[C@H]1/C(=C/CO)O[C@@H]2CC(=O)N21]

The inventive composition also includes amoxicillin, which is an analog of ampicillin, derived from the basic penicillin nucleus 6-aminopenicillanic acid. Chemically, a=Amoxicillin is (2S,5R,6R)-6-[I-(−)-2-amino-2-(p-hydroxyphenyl)acetamido]-3,3-dimet-7-oxo-4-thia-1-azabicyclo[3.2.0]heptane-2-carboxylic acid.

The composition of the invention contains no more than 21.5 mg/5 mL of clavulanate potassium, with amoxicillin within the inventive composition. Preferably the clavulanate potassium is present in an amount of between about 5 mg/5 mL to about 21.5 mg/5 mL.

A particularly preferred mode of administration for use with children is orally via an aqueous suspension. For preparing such suspensions amoxicillin and clavulanate potassium can be combined with buffers, emulsifying and suspending agents. If desired, certain sweetening and/or flavoring agents can be added. These active compounds can be directly mixed with liquid ingredients to provide a suspension, or can be formed into granules or powders which are then made into a suspension, by known methods and using known ingredients examples of which are provided below. The resulting suspension can be stored in the presence of water, especially if refrigerated, for an appropriate period. However, a preferred method is to store the mixture as a dry powder until its use is required, at which time it is mixed with an appropriate diluent, e.g., water.

The prescribing physician will ultimately determine the appropriate dose for a given human pediatric subject, and this can be expected to vary according to the age, weight, and response of the individual pediatric patient as well as the nature and severity of the patient's symptoms. The compounds of this invention will normally be used orally at dosages in the range from about 1.66 mg/kg/day to about 3.2 mg/kg/day clavulanate potassium for children weighing less than 40 kg. In some instances, it can be necessary to use doses outside these ranges.

Dosage forms contemplated for the compositions of this invention containing amoxicillin and clavulanate potassium, include any known liquids for pharmaceutical use, preferably oral suspensions as the typical patient will be a human child. The most common formulation is a powder for suspension to be mixed with water at the time of use.

The inventive composition can also contain excipients, vehicles, and solvents include sterile water, saline, Ringer's solution, polyalkylene glycols, natural and synthetic fatty acids, mono, di, and triglycerides and oils, and hydrogenated naphthalenes. Carriers can be included such as but not limited to lactose, saccharose, sorbitol, mannitol, starch, amylopectin, cellulose derivatives, and gelatin.

Disintegrants can be included such as but not limited to starch such as pregelatinized and sodium starch glycolate, cellulose such as microcrystalline, sodium carboxymethyl, hydroxypropyl, croscarmellose sodium, crosspovidone, and crosslinked polyvinyl pyrrolidone. Fillers can be included such as but not limited to cellulose, dibasic calcium phosphate, lactose, sucrose, glucose, mannitol, sorbitol, calcium carbonate, and fats and oils for capsules.

Antifriction agents can be included such as but not limited to magnesium and calcium stearates, and polyethylene glycol waxes. Glidants can be included such as but not limited to colloidal silicon dioxide and talc. Lubricants can be included such as but not limited to talc, silica, colloidal silicon dioxide, and fats such as zinc or magnesium stearate or stearic acid. Preservatives can be included such as but not limited to e m-cresol, p-cresol, o-cresol, chlorocresol, benzyl alcohol, phenylmercuric nitrite, phenoxyethanol, formaldehyde, chlorobutanol, magnesium chloride (hexahydrate), benzalkonium chloride, benzethonium chloride, sodium dehydroacetate, thimerosal, antioxidants (vitamins A, C, E, retinyl palmitate), selenium, cysteine, methionine, citric acid, sodium citrate, and lower alkylparabens. Mucoadhesives can be included such as but not limited to methyl, hydroxypropyl, and sodium carboxymethyl cellulose, chitosan, polyvinyl pyrrolidone, and hydrogels.

Binders can be included such as, but not limited to, polyvinylpyrrolidone, pregelatinized starch, methacrylic acid polymers, gelatin, and hydroxypropylcellulose. pH modifiers can be included such as but not limited to various organic and inorganic acids, bases, and their salts such as orthophosphoric acid, hydrochloric acid, nitric acid, sulphuric acid, sulfamic acid, hydrofluoric acid, oxoacids, sodium and potassium dihydrogen phosphates, citric acid, ascorbic acid, tartaric acid, malic acid, malonic acid, succinic acid, fumaric acid, maleic acid, adipic acid, lactic acid, levulinic acid, sorbic acid, polyacrylic acid, sodium carbonate, sodium bicarbonate, magnesium carbonate, magnesium oxide, calcium carbonate, calcium oxide, aluminum hydroxide, magnesium hydroxide, and sodium hydroxide.

Buffers can be included such as but not limited to acetic acid, citric acid, boric acid, and phosphoric acid. Isotonicity agents can be included such as but not limited to glycerin, mannitol, sorbitol, sodium chloride, and other electrolytes. Emulsifiers can be included such as but not limited to soy lecithin, calcium stearoyl dilactate, various esters of polyglycerol and sorbitan, and monoglycerides. Suspending agents can be included such as but not limited to natural and synthetic polysaccharides such as gums (acacia, tragacanth, guar, and xanthan), celluloses (sodium carboxymethyl, methyl, hydroxyethyl, hydroxypropyl, and microcrystalline), cargeenan, sodium alginate, carbomer, colloidal silicon dioxide, and clays (aluminum magnesium silicate, bentonite, hectorite).

Further components such as solubilizers can be added including but not limited to Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) and non-ionic surfactants such as polysorbate 20 or 80 or poloxamer 184 or 188, polyols, other block co-polymers, and chelators such as EDTA and EGTA.

Flavorants can be included such as but not limited to sodium saccharin, sugar, and other natural and artificial compounds which mask or enhance flavor. Colorants can be included such as but not limited to natural dyes such as caramel coloring, annatto, cochineal, betanin, turmeric, saffron, paprika, elderberry, pandan, and butterfly pea, and artificial dyes such as FD&C Blue Nos. 1 and 2, Green No. 3, Yellow Nos. 5 and 6, and Red Nos. 3 and 40. Thickening agents can be included such as but not limited to alginic acid and salts (such as sodium, potassium, ammonium, calcium), agar, carrageenan, locust bean gum, gelatin, and pectin. Stabilizing agents can be included such as but not limited to fatty acid salts, sulfates, sulfate esters and phosphate esters (for example polyoxyethylene fatty acid esters and alcohols, and polyoxyethylene sorbitol fatty acid esters such as polyoxyethylene sorbitan monooleate, polysorbate 80 and polysorbate 20). Surfactants can be included such as but not limited to sorbitan trioleate, soya lecithin, and oleic acid.

The sweetener of the composition can be any natural or synthetic compound, or combination of compounds, which provides adequate sweetening to overcome the bitterness of the inventive composition. Natural sweeteners include carbohydrates such as sucrose, dextrose, fructose, invert sugar, mannitol, sorbitol, and the like. Synthetic sweeteners include saccharin, aspartame, cyclamates, and other so-called artificial sweeteners familiar to those of skill in the art. The flavoring of the composition can be any natural or synthetic compound, or combination of compounds, which provides acceptable taste to overcome the blandness of the base composition. Such flavorings include bubble gum, grape, cherry, berry, citrus, other fruits, peppermint, spearmint, other mints, vanilla, chocolate, and the like, familiar to those of skill in the art.

In more detail, the compositions of this invention can be liquid formulations for oral use. Such formulations can include a suitable selection of appropriate known ingredients such as those provided above alone or in combination. The liquid formulations can be formulated as syrups, solutions or emulsion, elixir, suspensions or other known types of liquid suitable for oral administration. The liquid formulations can be aqueous or nonaqueous and include for example buffers with any pharmaceutically acceptable salts, preservatives, emulsifiers, humidifiers, isotonicity agents, solubilizers, buffers, thickening and suspending agents, dyes, and flavorants.

Specific ingredients can include water, saline, polyalkylene glycols, oils, hydrogenated naphthalenes, sugar, ethanol, glycerol, propylene glycol, dyes, flavorants, and thickening agents. The liquid formulations can be prepared by known methods using the compositions of this invention. The active compounds of the amoxicillin and clavulanate potassium can be obtained from existing powders, granules, or tablets for liquid formulations.

The liquid formulations of this invention can be provided for oral administration. For example, the formulations can be taken in measured doses using a cup, straw, spoon, syringe, or other device. The formulations can be provided in liquid form, or can be provided in dry form (for example granule or powder) to which an appropriately formulated water or liquid solvent is added to provide a liquid formulation of a composition of this invention. Ingredients suitable for liquid formulations are known and such a formulation can be made by methods known in the art.

A liquid formulation (for example solution, suspension, emulsion) can be made by combining the amoxicillin and clavulanate potassium with suitable aqueous and or nonaqueous diluents, water, buffers, and preservatives as discussed above and mixing with known methods under suitable known conditions. The specific ingredients and concentrations will depend on the type of formulation desired, for example oral suspension as known in the art. However, as an example, a pediatric oral suspension can also include a vehicle such as water, saline, Ringer's solution, dextrose, serum albumin, sodium chloride, mannitol, buffers, and preservatives. The formulation can be sterilized by known techniques.

### Therapeutic Methods

Certain known dosing combinations of amoxicillin and clavulanate potassium are listed in Table 1 below. In these conventional dosing combinations, the amount of clavulanate potassium is between about 28.5 mg/5 mL to about 62.5 mg/5 mL. The previous known and used ratios of amoxicillin to clavulanate potassium are between 4:1 to 14:1.

TABLE 1
Amoxocillin and clavulanate amounts and ratios.

| | Amoxicillin | Clavulanate potassium | Ratio |
|---|---|---|---|
| 1 | 125 mg/5 mL | 31.25 | 4:1 |
| 2 | 200 mg/5 mL | 28.5 | 7:1 |
| 3 | 250 mg/5 mL | 62.5 | 4:1 |
| 4 | 400 mg/5 mL | 57 | 7:1 |
| 5 | 600 mg/5 mL | 42.9 | 14:1 |

The inventive composition instead contains amoxicillin and clavulanate potassium wherein the amount of clavulanate potassium does not exceed 21.5 mg/5 mL. Further, another embodiment of the inventive composition contains amoxicillin and clavulanate potassium in a ratio of at least 26:1 and preferably between 28:1 and 56:1. In some embodiments, the ratio of amoxicillin to clavulanate potassium is at least 28.17:1, at least 31.69:1, 48:1, or at least 54:1. Disclosed embodiments of the invention result in a reduced amount of clavulanate potassium compared to current known compositions. While not being bound by theory, the reduced amount of clavulanate potassium can be especially good for pediatric patients as it can lead to reduced chance of diarrhea, vomiting, and diaper rash in such patients. The efficacy of amoxicillin for treating acute otitis media or other illnesses such as respiratory illnesses can be unaffected by the reduced amounts of clavulanate potassium.

Disclosed herein are methods to treat pediatric patients with a dosing regimen of about one to about fourteen days, using a composition of amoxicillin and clavulanate potassium wherein the clavulanate potassium is present in an amount not to exceed 21.5 mg/5 mL. In some embodiments of the method, the amount of clavulanate potassium remains constant, with the amount being less than 21.5 mg/5 mL.

In some embodiments, the first administered dose contains more clavulanate potassium than a second administered dose, or one or more subsequent dosages. For example, in a multiple day method spanning ten (10) days, the dosage of clavulanate potassium on days 1 and 2 can be in an amount of 21.5 mg/5 mL, then an amount of 15 mg/5 mL on days 3-10.

A further embodiment of the method of the present invention includes a dosage for the first two days of the treatment containing no more than 21.5 mg/5 mL of clavulanate potassium and then the subsequent dosages contain a less amount of clavulanate potassium. In yet another embodiment of the method of the present invention the first dose contains more of the clavulanate potassium than the second dose, and the subsequent dosages contain decreasing amounts of clavulanate potassium. The reduced amount of clavulanate potassium can be constant or can continue to decrease over the treatment days. For example, in a multiple day method spanning ten (10) days, the dosage of clavulanate potassium on days 1 and 2 can be in an amount of 21.5 mg/5 mL, then an amount of about 15-20 mg/5 mL on days 3-6, and an amount of about 5-10 mg/5 mL on days 7-10. In all the embodiments of the inventive methods, the amounts used should be effective for the treatment contemplated, as can be determined by a person skilled in the art. For example, the can comprise from about 1.66 mg/kg/day to about 3.2 mg/kg/day clavulanate potassium for children age two or less.

In another embodiment of the inventive method, the method of the invention is to treat pediatric patients with a dosing regimen of one to about fourteen days, using a composition of amoxicillin and clavulanate potassium in a ratio of at least 26:1 or at least 28:1. In another embodiment of the method of the present invention it is preferred that the first dose contains more of clavulanate potassium, than the second, and any subsequent dosages contain decreasing amounts of clavulanate potassium, so that the ratio increases over the dosage treatment regimen. The amounts used should be effective for the treatment contemplated, as can be determined by a person skilled in the art. For example, the can comprise from about 1.66 mg/kg/day to about 3.2 mg/kg/day clavulanate potassium for children age two or less.

The amounts of the amoxicillin in either embodiment of the inventive method, will preferably remain the same during the treatment regimen, but can also decrease, or can even increase for any treatment that would require increasing amounts. The amounts of the clavulanate potassium can decrease with each administration or can remain constant over several administrations, or even increase if the treatment requires. It is most preferable that the amount of the clavulanate potassium, decreases with successive administrations. The dosages can be formulated to contain more than one unit dose and thus be administered more than once. Thus it is possible that two or more successive dosages as administered can contain the same amount of the clavulanate potassium, while the next subsequent dosage contains less. The dosages can also be provided as a unit dose, in which case each is only provided once. Any combination of dosages and unit doses can be used. One embodiment of the method is for a multiple day administration wherein the clavulanate potassium is a constant amount over the treatment days, with the days being anywhere from about two days to about fourteen days or more. Another embodiment of the method is for a multiple day administration wherein the clavulanate potassium is reduced over the administration period. The administration period can also be about two days or increased up to about fourteen days or more, with either constant or reduced clavulanate potassium over the administration period.

For instance, the amount or concentration of the clavulanate potassium can remain constant in the ratio to amoxicillin of at least 26:1 or at least 28:1 amoxicillin:clavulanate potassium, or could taper down in succeeding dosages of the method. In the reducing embodiment, thus each composition will contain less of the clavulanate potassium and the ratio will increase. The final dosing composition can optionally contain none of the clavulanate potassium.

In general the compositions of this invention contain effective amounts for the treatment contemplated of the amoxicillin and clavulanate potassium. These amounts can be determined by a skilled person with routine experimentation.

However, preferred amounts of amoxicillin and clavulanate potassium are provided as follows. In any liquid formulations of the compositions of this invention described above, preferably suspensions, each composition can contain from about 100 to about 1200 mg/5 mL (or about 20 to about 140 mg/mL) of amoxicillin and about 0.1 to about 21.5 mg/5 mL of clavulanate potassium. The compositions preferably contain from about 125 mg/6 mL to about 600 mg/5 mL of amoxicillin. Preferably the amount of clavulanate potassium is from about 0.01 mg/5 mL to about 21.5 mg/5 mL of liquid formulation. The amount of clavulanate potassium can be about 22.6 mg/5 mL. For purposes of dosing 5 mL is equal to one teaspoon.

For any given dispensing system of this invention, the amount of clavulanate potassium in the first composition should preferably determine the amount in the second (and succeeding) compositions in an embodiment wherein there will be the same or less clavulanate potassium in succeeding doses. For example, if the first composition contains 600 mg/5 mL of amoxicillin and 21.5 mg/5 mL of clavulanate potassium, then the second dosing composition contains equal to or less than 21.5 mg/5 mL clavulanate potassium.

This invention is also directed to a liquid composition of amoxicillin and clavulanate potassium which contains less than about 21.5 mg/5 mL and more than 0.1 mg/5 mL of clavulanate potassium. A preferred composition has from about 15 mg/5 mL to about 21.5 mg/5 mL. Another preferred composition has an amount of clavulanate potassium of about 22.6 mg/5 mL Another preferred composition has from about 10 mg/5 mL to less than 21.5 mg/5 mL. Another preferred range is from about 2.5 mg to about 10 mg of clavulanate potassium.

The concentrations of the invention are preferably expressed in mg as above. This invention also contemplates ratios to express the concentrations. For example, the amoxicillin can be present in a ratio of about 28:1 to about 56:1 where 1 represents the amount of the clavulanate potassium. Preferably the amount of the amoxicillin is from about 30:1 to about 35:1. These ratios are preferably weight ratios. As discussed above, compositions of this invention can start with any amount or concentration of the clavulanate potassium as long as the succeeding compositions contain the same or lower amounts or concentrations.

The amounts and concentrations of the pharmaceutically active compounds, preferably amoxicillin and clavulanate potassium, can also be determined by known methods using desired serum concentrations at various points in the treatment regimen.

The compositions of the present invention can be prepared by known processes. Amoxicillin and clavulanate potassium, the preferred pharmaceutically active compounds can be obtained from suppliers or made by known methods. See for example U.S. Pat. Nos. 6,218,380 and 7,534,781.

The formulations discussed above can be made by methods known in the art using the various “inactive” formulation ingredients discussed with amoxicillin and clavulanate potassium. These known ingredients can be made by methods known in the art or obtained from chemical supply houses. The amounts and concentrations preferred for the amoxicillin and clavulanate potassium compositions of this invention are discussed above. The amounts of the other ingredients should be sufficient to provide the properties for which each of the ingredients are being used, for example, flavorant or other additives.

Liquid formulations of compositions of this invention can be prepared by mixing the pharmaceutically active compounds preferably amoxicillin and clavulanate potassium with a preservative and any desired buffers in an aqueous diluent using conventional procedures for mixing, suspension or dissolution. Liquid formulations can be made by reconstituting powders or granules or lyophilized preparations.

Suspensions of this invention can be provided at any concentration providing acceptable stability for the pharmaceutically active compounds (for example the length of the desired treatment period, optionally with refrigeration) and within the range that would provide a composition having suitable flow parameters for dispensing systems of this invention. Reconstituting oral suspensions from an amoxicillin and clavulanate potassium powder composition can be done as follows from a powder prepared for oral suspension. The suspension can be prepared from freely flowing powder in a suitable container. A little over half of the solvent such as water needed should be added and the container shaken vigorously to suspend. Then the rest of the solvent should be added and the container shaken vigorously.

The preferred active ingredients are amoxicillin and clavulanate potassium used together in the treatment regimen described, however other active ingredients can be used in the same type of composition of this invention. Further, the amoxicillin and clavulanate potassium can be present throughout the multiple day dosing method in a constant amount of about 21.5 mg/5 mL or less of clavulanate potassium to at least 600 mg/5 mL, or more of amoxicillin, in a ratio of at least about 28:1.

Any pharmaceutically acceptable formulation of the compositions of this invention including the amoxicillin and clavulanate potassium at a ratio of at least about 28:1 can be used in the dispensing systems of this invention. Such compositions can contain pharmaceutically acceptable ingredients whose nature and amounts will be known to a skilled practitioner depending on the dosage form and route of administration selected. Amoxicillin and clavulanate in any pharmaceutically acceptable form can be used in any combinations, including salts, complexes, prodrugs, hydrates, solvates, or polymorphs. Clavulanate potassium is preferred. Other pharmaceutically active ingredients can also be included in the compositions of this invention.

This invention is directed to a method of treatment by providing two or more doses of a composition containing amoxicillin and clavulanate potassium. A preferable condition to be treated is a bacterial infection, most preferably acute otitis media. Other conditions for treatment include respiratory bacteria illness such as sinusitis.

The patient or treatment subject can be a human, preferably a human child. Other patients can include non-humans such as animals. Therefore, the method can include a veterinary method to treat infections, viruses and bacteria in mammals, fish, birds and animals.

The amount of the compounds used in the inventive composition and method of treatment are amounts effective to treat the condition. More specific amounts have been discussed in detail above. The dosage will depend on the age, weight, condition, and disease of the patient. In general, the compositions of this invention contain effective amounts for the treatment contemplated of the amoxicillin and clavulanate potassium. These amounts can be determined by a skilled person with routine experimentation.

The method of the claimed invention can include two or more administrations of amoxicillin combined with clavulanate potassium where the amounts remain constant or the initial administration contains more or less clavulanate potassium than any subsequent administrations. The clavulanate potassium amount can be constant throughout the dosage, with the amount being less than conventional dosages, such as less than about 21.5 mg/5 mL. In another embodiment, the initial dose can be a unit dose which contains more clavulanate potassium than the second dose which is a unit dose and any subsequent unit doses. The initial administration can be more than one unit dose containing more clavulanate potassium than the second and subsequent administrations which also can include more than one unit dose. Similarly, the first administration can be a unit dose and subsequent administrations include more than one unit dose. The first administration can be more than one dose and second and/or subsequent doses can be unit doses. The distinction between the first and subsequent administrations is an embodiment of this invention.

In a further embodiment of the inventive method, the treatment regimen can be over multiple days where the initial administration amount of clavulanate potassium is about or less than about 21.5 mg/5 mL, the middle administrations are a higher amount than the first while still being less than about 21.5 mg/5 mL, and the next subsequent administrations are in an amount lower than the middle administrations. For example, in a multiple day method spanning ten (10) days, the administration of clavulanate potassium on days 1 and 2 can be in an amount of about 10-19 mg/5 mL, then an amount of about 20-21.5 mg/5 mL on days 3-6, and an amount of about 5-10 mg/5 mL on days 7-10.

The compositions of this invention and the methods of this invention can be used to provide various treatment regimens to patients as methods of treatment of this invention. A method of treatment regimen of the present invention can be one day or multiple days, between about two days to about fourteen days, though it is preferred the dosing be for about three through about ten days. The dosage schedule below are given solely as examples; many others can readily be developed by a skilled practitioner based on known methods and information provided herein.

For example, a treatment period of about ten days can comprise providing amoxicillin and clavulanate potassium wherein the dosing remains constant of amoxicillin and clavulanate potassium where the clavulanate is in an amount of about 21.5 mg/5 mL or less.

Another example of a treatment period of ten days can include the following dosing as follows:

Days 1-4: 600 mg/kg amoxicillin and 21.5 mg/kg clavulanate potassium; and

Days 5-10: 600 mg/kg amoxicillin and 10.75 mg/kg clavulanate potassium, should provide suitable dosages for a pediatric patient.

In another embodiment a treatment period of ten days providing amoxicillin and clavulanate potassium wherein the dosing remains constant of amoxicillin and clavulanate potassium where the clavulanate potassium is present in an amount of about 21.5 mg/5 mL or less.

Another example of a treatment period of ten days can include the following dosing as follows:

Days 1-2: 600 mg/kg amoxicillin and 21.5 mg/kg clavulanate potassium;

Days 3-5: 600 mg/kg amoxicillin and 10.75 mg/kg clavulanate potassium; and

Days 6-10: 600 mg/kg amoxicillin and 5.5 mg/kg clavulanate potassium, should provide suitable dosages for a pediatric patient.

As can be seen this exemplary regimen can be modified for different formulations, reduced or extended in length, and designed to provide further clavulanate potassium gradients if desired, by varying the amounts and concentrations of the compositions of this invention and selecting the appropriate dispensing system of this invention. Other examples will be apparent to a skilled practitioner and are part of this invention.

Preferably the amount of clavulanate potassium in subsequent dosages is from about 0.1 mg/5 mL of suspension to about 21.5 mg/5 mL of suspension. More preferably the amount of clavulanate potassium is from about 10 mg/5 mL of suspension to about 21.5 mg/5 mL of suspension.

In general, this invention provides methods of treatment as discussed above for infections in a patient, of any part of the body including specific cells, tissues, or organs. The infections can be acute or chronic and are primarily bacterial such as meningitis, peritonitis, Chlamydia pneumoniae, S. pneumoniae, listeriosis, salmonellosis, toxic shock syndrome, tuberculosis, and other bacterial infections. Syndromes and conditions caused by bacterial infections can also be treated, such as hemolytic uremic syndrome and Lyme disease.

Bacterial infections for treatment with the compositions of this infection include but are not limited to acute otitis media and other infections such as those of the lower respiratory tract, sinusitis, skin and skin structure infections and urinary tract infections.

These can be caused by caused by various bacteria both Gram-positive and Gram-negative. Among them are Staphylococcus aureus, Enterobacter species in urinary tract infections, Escherichia coli, H. influenzae, M. catarrhalis, S. pneumoniae, Neisseria gonorrhoeae, Eikenella corrodens, Proteus mirabilis, Enterococcus faecalis, Staphylococcus epidermidis, Staphylococcus saprophyticus, Streptococcus pyogenes, viridans group streptococcus, Klebsiella species Bacteroides species, Fusobacterium species, and Peptostreptococcus species.

As discussed above there are various formulations and dispensing methods for compositions of this invention. The appropriate dosages can be determined as discussed above within the bounds of this invention regarding initial and subsequent doses with regard to the relative amounts of the amoxicillin and clavulanate potassium. Delivery methods include but are not limited to liquid and oral suspensions. Dispensing systems include containers, syringes, spoons, straws and the like.

The inventive pediatric oral suspension composition containing the reduced amount of clavulanate potassium compared to conventional compositions should correspondingly reduce possible less common but severe side effects of jaundice and hepatitis, hemorrhagic/pseudomembranous colitis, angioedema, Stevens-Johnson syndrome, hypersensitivity vasculitis, tooth discoloration, and seizure, as well as the more common and disruptive side effects of diarrhea, vomiting or diaper rash, headache, mycosis, vaginitis and agitation, all while still maintaining the efficacy and benefits of the antibiotic treatment for acute otitis media and other illnesses. This should be seen whether the clavulanate potassium remains in a constant dosage throughout the about one to about fourteen day treatment regimen method, of less than about 21.5 mg/5 mL, or if the clavulanate potassium dosage is reduced through the respective about one day to about fourteen day dosage, being reduced either once, more than once or with each subsequent unit dose.

The present invention thus can comprise a pediatric oral suspension composition having amoxicillin-clavulanate potassium of minimally sufficient quantity so as to maintain the efficacy of the composition in view of beta-lactamase mediated resistance H. influenzae and M. catarrhalis, without elevating the possibility of the severe side effects of jaundice and hepatitis and the more common and disruptive side effects of diarrhea, diaper rash and vomiting. The composition allows the amoxicillin to be used as intended while reducing the side effects of the clavulanate potassium while further still maintaining the efficacy of the overall composition when dealing with various beta-lactamase medicated resistance issues.

Further, the reduced dosage treatment of the present invention can either be maintained throughout the treatment or further reduced throughout the treatments over subsequent days. Again, this reduced amount of clavulanate potassium in the total composition can be constant throughout the treatment regimen or preferably reduced throughout the treatment regimen, with the amount being of minimal sufficient quantity so as to maintain the efficacy of the total composition in view of beta-lactamase mediated resistance H. influenzae and M. catarrhalis.

Also disclosed herein are methods of treating pediatric otitis media in a patient under 24 months of age, the method comprising administering amoxicillin and clavulanate potassium to the patient in dosages of at least about 40 mg/kg/day amoxicillin and from about 1.66 mg/kg/day to about 2.99 mg/kg/day clavulanate potassium. In some embodiments, the dosage is achieved with two or more daily administrations.

The methods can comprise administering amoxicillin in any herein disclosed amount, but at least about 40 mg/kg/day amoxicillin is used. In some embodiments, at least 45 mg/kg/day, or at least 50 mg/kg/day of amoxicillin are administered. In some embodiments, the methods comprise administering amoxicillin in a range from about 40 mg/kg/day to about 90 mg/kg/day, from about 45 mg/kg/day to about 90 mg/kg/day, from about 50 mg/kg/day to about 90 mg/kg/day, from about 60 mg/kg/day to about 90 mg/kg/day, from about 70 mg/kg/day to about 90 mg/kg/day, or from about 80 mg/kg/day to about 90 mg/kg/day. In some embodiments, about 80 mg/kg/day, about 81 mg/kg/day, about 82 mg/kg/day, about 83 mg/kg/day, about 84 mg/kg/day, about 85 mg/kg/day, about 86 mg/kg/day, about 87 mg/kg/day, about 88 mg/kg/day, about 89 mg/kg/day, or about 90 mg/kg/day of amoxicillin are administered. In some embodiments, the methods comprise administering amoxicillin in a range from about 40 mg/kg/day to about 50 mg/kg/day, from about 40 mg/kg/day to about 45 mg/kg/day, or from about 45 mg/kg/day to about 50 mg/kg/day. In some embodiments, about 40 mg/kg/day, about 41 mg/kg/day, about 42 mg/kg/day, about 43 mg/kg/day, 44 mg/kg/day, about 45 mg/kg/day, about 46 mg/kg/day, about 47 mg/kg/day, 48 mg/kg/day, about 49 mg/kg/day, or about 50 mg/kg/day of amoxicillin are administered. It is expressly understood that the methods can comprise administering amoxicillin in a dosage ranging from any lower amount to any higher amount of the aforementioned dosage amounts. For example, and without limitation, the methods can comprise administering amoxicillin in a dosage ranging from about 83 mg/kg/day to about 89 mg/kg/day, or from about 81 mg/kg/day to about 86 mg/kg/day.

The methods can comprise administering from about 1.66 mg/kg/day to about 2.99 mg/kg/day clavulanate potassium. It was a surprising finding that dosages containing very small amounts of clavulanate, for example from 1.66 mg/kg/day to 2.99 mg/kg/day, could reduce side effects while maintaining therapeutic effectiveness. It was also surprising that such dosages of clavulanate were also effective in combination with lower dosages of amoxicillin, for example as low as about 40 mg/kg/day amoxicillin, or about 80 mg/kg/day amoxicillin, or from about 80 mg/kg/day to about 90 mg/kg/day amoxicillin.

In some embodiments, the methods can comprise administering from about 1.66 mg/kg/day to about 2.84 mg/kg/day clavulanate potassium. In some embodiments, the methods can comprise administering from about 1.66 mg/kg/day to about 2.75 mg/kg/day, from about 1.66 mg/kg/day to about 2.5 mg/kg/day, from about 1.66 mg/kg/day to about 2.25 mg/kg/day, from about 1.66 mg/kg/day to about 2.0 mg/kg/day, or from about 1.66 mg/kg/day to about 1.8 mg/kg/day clavulanate potassium. In some embodiments, the methods can comprise administering clavulanate potassium in a dosage of about 1.66 mg/kg/day, about 1.67 mg/kg/day, about 1.68 mg/kg/day, about 1.69 mg/kg/day, about 1.7 mg/kg/day, about 1.8 mg/kg/day, about 1.9 mg/kg/day, about 2.0 mg/kg/day, about 2.1 mg/kg/day, about 2.2 mg/kg/day, about 2.3 mg/kg/day, about 2.4 mg/kg/day, about 2.5 mg/kg/day, about 2.6 mg/kg/day, about 2.7 mg/kg/day, about 2.8 mg/kg/day, about 2.9 mg/kg/day, or about 2.99 mg/kg/day. In some embodiments, the methods can comprise administering from about 2.85 mg/kg/day to about 3.2 mg/kg/day clavulanate potassium. In some embodiments, the methods can comprise administering from about 2.95 mg/kg/day to about 3.2 mg/kg/day, from about 3.0 mg/kg/day to about 3.2 mg/kg/day, or from about 3.1 mg/kg/day to about 3.2 mg/kg/day of clavulanate potassium. In some embodiments, the methods can comprise administering clavulanate potassium in a dosage of about 2.85, about 2.86, about 2.88, about 2.90, about 2.92, about 2.94, about 2.95, about 2.96, about 2.98, about 3.0, about 3.02, about 3.04, about 3.05, about 3.06, about 3.08, about 3.1, about 3.12, about 3.14, about 3.15, about 3.16, about 3.18, or about 3.2 mg/kg/day. It is expressly understood that the methods can comprise administering clavulanate potassium in a dosage ranging from any lower amount to any higher amount of the aforementioned dosage amounts. For example, and without limitation, the methods can comprise administering clavulanate potassium in a dosage ranging from about 1.7 mg/kg/day to about 2.8 mg/kg/day, or from about 1.66 mg/kg/day to about 2.7 mg/kg/day.

The methods can comprise administering 2.85 mg/kg/day to about 3.2 mg/kg/day clavulanate potassium. It was a surprising finding that dosages containing very small amounts of clavulanate, for example from 2.85 mg/kg/day to about 3.2 mg/kg/day, could reduce side effects while maintaining therapeutic effectiveness when combined with amoxicillin.

Amoxicillin and clavulanate potassium can be administered in a ratio of at least 15:1, or between 15:1 and 56:1. In some embodiments, the ratio of amoxicillin to clavulanate potassium is at least 18:1, at least 20:1, at least 25:1, at least 26.8:1, at least 28.17:1, at least 30.1:1, at least 31.69:1, at least 32:1, at least 36:1, at least 40:1, at least 45:1, at least 48:1, or at least 54:1. It is expressly understood that the methods can comprise administering amoxicillin and clavulanate potassium in a ratio ranging from any lower ratio to any higher ratio of the aforementioned ratios. For example, and without limitation, the methods can comprise administering amoxicillin and clavulanate potassium in a ratio ranging from about 28.17:1 to about at least 40:1, or from about 32:1 to about 48:1.

Amoxicillin and clavulanate potassium can be administered separately or, alternatively, in a single composition. Likewise, subsequent dosages can comprise separately administering amoxicillin and clavulanate potassium, or alternatively, administering amoxicillin and clavulanate potassium in a single composition.

The methods can include any of the herein disclosed dosing schedules. For example, and without limitation, the methods can comprise a first daily administration comprising more clavulanate potassium than a second daily administration. As another non-limiting example, the methods can comprise administering the daily dosages for fourteen days or less. In some embodiments, the total daily dose is provided in two or more daily administrations.

The methods can further comprise administering one or more additional therapeutics. The additional therapeutic can be administered with the amoxicillin, the clavulanate potassium, or both, in one or more dosages. In some embodiments, the additional therapeutic comprises a pain reduction medication, for example and without limitation, acetaminophen, a non-steroidal anti-inflammatory medication, or an antipyretic medication.

The patient can be any herein disclosed patient under 24 months of age. In some embodiments, the patient has had frequent exposure to other children. In some embodiments, the patient has been previously treated with one or more antibiotics.

The methods can provide beneficial therapeutic effects for a patient administered according to the disclosed methods. In some embodiments, the method reduces a rate of protocol-defined diarrhea (PDD) by at least 9 percent compared to a control treatment. In some embodiments, the method reduces a rate of diaper dermatitis by at least 8 percent compared to a control treatment.

The inventive compositions and methods can be compared to a control. The control can be a treatment comprise administering two or more dosages of an oral suspension comprising about 90 mg/kg/day amoxicillin and about 6.4 mg/kg/day clavulanate potassium for ten days. The control need not be experimentally performed with the methods and can alternatively be a collection of values used as a standard applied to one or more subjects (e.g., a general number or average that is known and not identified in the method using a sample).

Also disclosed are methods of treating a pediatric patient under 24 months of age for a drug resistant bacterial infection, the method comprising administering an oral suspension comprising amoxicillin and clavulanate potassium to the patient in two or more dosages comprising at least about 40 mg/kg/day of the amoxicillin and from about 1.66 mg/kg/day to about 2.84 mg/kg/day of the clavulanate potassium. In some embodiments, at least about 80 mg/kg/day of the amoxicillin are administered.

The methods are effective for treating an array of bacterial infections, which can include but are not limited to infections causing acute otitis media and other infections such as those of the lower respiratory tract, sinusitis, skin and skin structure infections and urinary tract infections. Such infections can be caused by various bacteria, both Gram-positive and Gram-negative. Among them are staphylococcus aureus, Enterobacter species in urinary tract infections, Escherichia coli, H. influenzae, M. catarrhalis, S. pneumoniae, Neisseria gonorrhoeae, Eikenella corrodens, Proteus mirabilis, Enterococcus faecalis, Staphylococcus epidermidis, Staphylococcus saprophyticus, Streptococcus pyogenes, viridans group streptococcus, Klebsiella species, Bacteroides species, Fusobacterium species, and Peptostreptococcus species. In some embodiments, the drug resistant bacterial infection can comprise a bacterial species that produces a β-lactamase. In some embodiments, the drug resistant bacterial infection can comprise Streptococcus pneumoniae, Haemophilus influenzae, Moraxella catarrhalis, or combinations thereof.

Also disclosed are methods of treating a beta-lactamase producing Haemophilus influenzae or Moraxella catarrhalis in a pediatric patient under 24 months of age, the method comprising administering amoxicillin and clavulanate potassium to the patient in dosages of at least about 80 mg/kg/day of the amoxicillin and from about 1.66 mg/kg/day to about 2.84 mg/kg/day of the clavulanate potassium.

Also disclosed are methods of treating pediatric otitis media in a patient under 24 months of age, the method comprising administering amoxicillin and clavulanate potassium to the patient wherein an amoxicillin dosage is from about 40 mg/kg/day to about 50 mg/kg/day, and a clavulanate potassium dosage is from about 2.85 mg/kg/day to about 3.2 mg/kg/day.

Also disclosed are methods of treating a pediatric patient under 24 months of age for a drug resistant bacterial infection, the method comprising administering amoxicillin and clavulanate potassium to the patient wherein an amoxicillin dosage is from about 40 mg/kg/day to about 50 mg/kg/day, and a clavulanate potassium dosage is from about 2.85 mg/kg/day to about 3.2 mg/kg/day.

Also disclosed are methods of treating a beta-lactamase producing Haemophilus influenzae or Moraxella catarrhalis in a pediatric patient under 24 months of age, the method comprising administering amoxicillin and clavulanate potassium to the patient wherein an amoxicillin dosage is from about 40 mg/kg/day to about 50 mg/kg/day, and a clavulanate potassium dosage is from about 2.85 mg/kg/day to about 3.2 mg/kg/day.

Having generally described the invention, the same will be more readily understood by reference to the following example, which is provided by way of illustration and are not intended as limiting.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Formulations

These formulations are provided as examples of possible oral suspension formulations of this invention. It will be apparent that these formulations and many variations of these formulations are available as compositions of this invention. Other types of formulations as discussed above such as aerosols, injectable solutions, capsules, topical formulations, and others can be included among these examples.

### Oral Suspension (Amounts in 5 mL)

200 mg Amoxicillin trihydrate and 3.36 mg (0.15 mEq) sodium
250 mg Amoxicillin trihydrate and 3.39 mg (0.15 mEq) sodium
400 mg Amoxicillin trihydrate and 4.33 mg (0.19 mEq) sodium
20 mg clavulanate potassium
10 mg clavulanate potassium
5 mg clavulanate potassium

### Oral Suspension:

200, 250, 400 mg Amoxicillin trihydrate
20, 10, 5 mg clavulanate potassium

### FD&C Red No 3

flavorings
silica gel
sodium benzoate
sodium citrate
sucrose
xanthan gum
These ingredients are sieved and milled separately and together, then blended and remilled, compacted by roller compaction, and screened with vibration to provide granules.

### Example 2: Assays

In order to confirm the effectiveness of the compositions of this invention, various biological assays can be performed. In vitro studies of various types known for antibiotic use can be used to demonstrate effectiveness and safety. Similarly in vivo studies in animal models can also be used for such purposes. Clinical studies are not required, but clinical data can be included to show the safety and effectiveness of the compositions of this invention.

Minimum inhibitory concentrations (MICs) or minimum bactericidal concentration (MBC) for relevant bacterial populations can be determined by known quantitative methods. The MIC is the lowest concentration that inhibits visible growth on a plate or reduces turbidity in culture and provide estimates of the susceptibility of given bacteria to the compositions tested. The MBC is the lowest concentration that kills almost all (99.9%) of the original culture in a given period of time.

For the MIC, the relevant bacteria (for example H. influenzae or S. pneumoniae) can be obtained from a culture collection such as the ATCC. The bacteria can be grown on nutrient agar plates. A disk diffusion test can then be performed. Paper disks containing amoxicillin-clavulanate potassium in different dilutions representing compositions are placed on the lawn of bacteria on the plate. Inhibition zones dependent on the effectiveness of the concentrations will appear and be measured for comparison with known standard zones for amoxicillin and clavulanate potassium. Zones equal to or greater than standard zones indicate extent of effectiveness.

For the MBC, the bacteria can be grown in the appropriate nutrient broth in test tubes. An MBC can be performed by introducing dilutions of a composition into tubes with the standardized inoculum of bacteria and comparing the turbidity by eye and by standard measuring procedures to determine the concentration at which there is minimal turbidity corresponding to 99.9% inhibition.

Using these tests, the effectiveness of the compositions of this invention can be demonstrated. One dilution pattern to be used would employ a constant concentration of amoxicillin combined with sequential dilutions of clavulanate potassium. The dilutions could begin at 20 mcg amoxicillin and 10 mcg clavulanate potassium per standard agar plate, and proceed by tenfold dilutions of clavulanate potassium, for example. MIC and MBC values can be measured by known methods and compared with standards, for example standards established for existing amoxicillin/clavulanate potassium combinations.

Further studies can be performed demonstrating the effectiveness of the compositions of this invention, for example MIC and bioavailability studies in mice. Such studies are well known and standardized. Different concentrations of the compositions of this invention could be administered to infected animals, in particular compositions with the same amount of amoxicillin and dilutions of clavulanate potassium, and the effectiveness determined by known methods.

### Example 3: Administration of 2.85-3.2 mg/kg/day Clavulanate

### Abstract

Amoxicillin/clavulanate (A/C), is currently the most effective oral antimicrobial in treating children with acute otitis media (AOM), but standard dosage of 90/6.4 mg/kg/day commonly causes diarrhea. An A/C formulation containing lower concentrations of clavulanate was examined to determine whether it could result in less diarrhea, while maintaining plasma levels of amoxicillin and clavulanate adequate to eradicate middle-ear pathogens and achieve clinical success.

An open-label study in children aged 6 to 23 months with AOM was conducted. In Phase 1, 40 children were treated with a reduced-clavulanate A/C formulation providing 90/3.2 mg/kg/day for 10 days. In Phase 2, 72 children were treated with the same formulation at a dosage of 80/2.85 mg/kg/day for 10 days. Children's rates of protocol-defined diarrhea (PDD), diaper dermatitis, and AOM clinical response were compared with rates reported in children who received the standard A/C regimen, and plasma levels of amoxicillin and clavulanate were obtained at various time points.

Some clinical outcomes in Phase 1 children and in children who received the standard regimen did not differ significantly. Rates of PDD in children receiving Phase 2 and standard regimens were 17% and 26%, respectively (P=0.10). Corresponding rates of diaper dermatitis were 22% and 34% (P=0.04), and of AOM treatment failure were 12% and 16% (P=0.44). Symptomatic responses did not differ significantly between regimens; both gave clavulanate levels sufficient to inhibit β-lactamase activity.

In young children with AOM, clavulanate dosages lower than those currently used may be associated with fewer side-effects without reducing clinical efficacy.

### Methods

Eligibility and Enrollment.

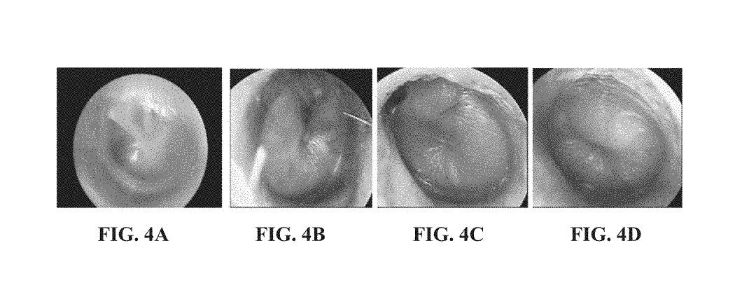

An open-label study was conducted between December 2015 and June 2016 at Children's Hospital of Pittsburgh of the University of Pittsburgh Medical Center (UPMC). The protocol was approved by the institutional review board; written informed consent was obtained from a parent of each enrolled child. To be eligible for enrollment, children were required to have AOM diagnosed on the basis of three criteria: onset of symptoms within the preceding 48 hours, a total score of 2 or more on the Acute Otitis Media-Severity of Symptoms (AOM-SOS) scale (Shaikh N, Hoberman A, Pediatr Infect Dis J 2009; 28:9-12; Shaikh N, Hoberman A, Paradise J L, et al. Pediatr Infect Dis J 2009; 28:5-8) middle-ear effusion; and moderate or marked bulging of the tympanic membrane (TM) or slight bulging accompanied by apparent otalgia or marked TM erythema (FIGS. 4A to 4D). Children were excluded who had TM perforation or another illness, were allergic to amoxicillin, or had received an antimicrobial within 96 hours. All study clinicians had successfully completed an otoscopic validation program.

Treatment.

In Phase 1, parents of 40 children were provided with a novel formulation of A/C containing a reduced concentration of clavulanate (600/21.5 mg/5 mL) that had been prepared at the UPMC Investigational Drug Service. The children were treated with 90 mg/kg/day and 3.2 mg/kg per day of the amoxicillin and clavulanate components, respectively, in two divided doses for 10 days. Because of the negative findings in Phase 1, 72 additional children were treated with the same reduced-clavulanate formulation (600/21.5 mg/5 mL), but dosed at 80/2.85 mg/kg/day in two divided doses for 10 days (Phase 2). For children's seeming discomfort, administration of acetaminophen was suggested.

Follow-up, Examination, Assessment and Management.

Children were assessed on a Day 7 visit and on an end-of-treatment visit, usually on Day 12 to 14. Parents were asked to record daily their children's AOM-SOS scores and the number and consistency of their bowel movements. On either the enrollment visit or the Day 7 visit, a single convenience sample of blood was obtained from each child whose parents gave consent. —Samples were obtained at time points ranging from 30 minutes to 4 hours after administration of study medication for determination of amoxicillin and clavulanate plasma levels. At the end-of-treatment visit, children were categorized as having experienced either clinical success or clinical failure, which was defined as worsening of symptoms or of otoscopic signs of infection (mainly TM bulging); or as failure to achieve, by the end of treatment, complete or nearly complete resolution of AOM-attributable symptoms and signs—without regard to persistence or resolution of middle-ear effusion. Children experiencing clinical failure were treated with a rescue regimen, consisting preferentially of amoxicillin-clavulanate 90/6.4 mg/kg/day for 10 days; or in children considered allergic to amoxicillin or whose response to amoxicillin-clavulanate was unsatisfactory, ceftriaxone, usually in 2 doses of 75 mg/kg each, administered intramuscularly 2 days apart.

Outcome Measures.

All outcome measures were pre-specified. The primary outcome was the proportion of children developing PDD, which was defined as the occurrence of three or more watery stools on 1 day or two or more watery stools on 2 consecutive days. Secondary measures included 1) a comparison of the aforementioned standard dosing regimen of amoxicillin/clavulanate (90/6.4 mg/kg/day for 10 days) vs. the reduced-clavulanate regimen used in Phase 2 (80/2.85 mg/kg/day for 10 days), regarding the proportion of children who experienced diaper dermatitis occasioning prescription of an antifungal cream; 2) clinical failure at the Day 12-14 visit; and 3) children's symptom burden over time, and the proportions of children whose symptomatic response considered satisfactory, defined as a greater than 50% decrease in AOM-SOS scores from baseline. (Shaikh N, Hoberman A, Rockette H E, Kurs-Lasky M, Paradise J L. J Pediatric Infect Dis Soc 2015; 4:367-9; Shaikh N, Rockette H E, Hoberman A, Kurs-Lasky M, Paradise J L. Pediatr Infect Dis J 2015; 34:e41-3). Study clinicians were asked to record at the Day 7 visit, based on each child's symptoms and otoscopic signs, their judgment regarding the suitability of discontinuing the child's antimicrobial treatment. To obtain a historical control group, the subgroups of children enrolled in two recent clinical trials (which had the same inclusion and exclusion criteria as in the present study) who were treated for 10 days with the standard A/C formulation and dosing regimen were combined (Hoberman A, Paradise J L, Rockette H E, et al. N Engl J Med 2011; 364:105-15; Hoberman A, Paradise J L, Rockette H E, et al. N Engl J Med 2016; 375:2446-56) At the end-of-treatment visit, parents were asked to rate their satisfaction with their children's therapy.

Statistical Analysis.

The proportion of children receiving a reduced-clavulanate formulation who developed PDD was hypothesized to be in the range of 10-20%. With a population of 75 children and applying a 90% confidence interval, the length of the confidence interval regarding the proportion of children developing PDD was calculated to be less than 0.12; applying an 80% confidence interval, the expected length was less than 0.08. For all analyses, present findings were compared with previously reported findings from use of the standard A/C formulation and dosing regimen as described above Logistic regression was used to compare the proportions of children who experienced adverse effects such as PDD and diaper dermatitis, the proportions experiencing clinical failure, and the proportions whose symptomatic response to treatment considered satisfactory as defined above. Time-to-event analysis was used to estimate the number of days until 15% of parents reported PDD in their children, and average AOM-SOS scores over specified periods were compared using generalized estimated equations.

### Results

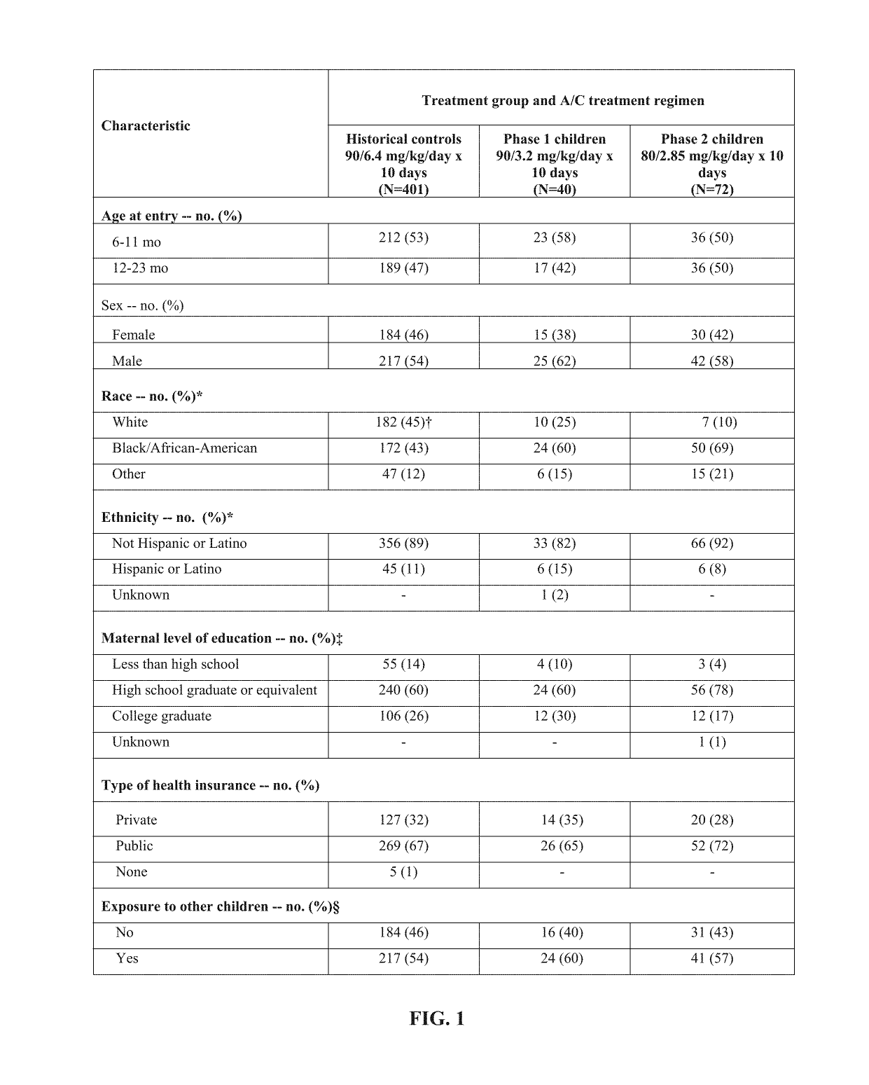

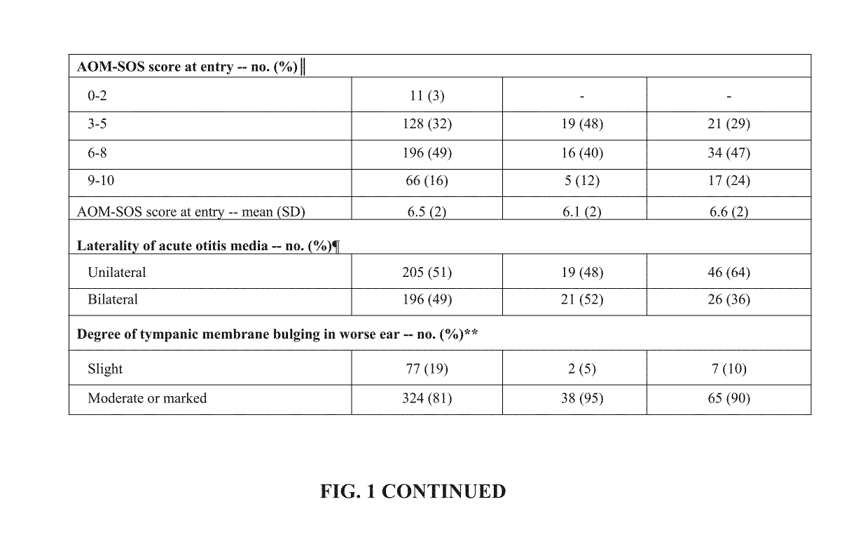

40 children were enrolled in Phase 1 and 72 children in Phase 2. Demographic and clinical characteristics of these children, compared with those of historical controls, are shown in FIG. 1; no differences, except as noted, were apparent.

Outcomes.

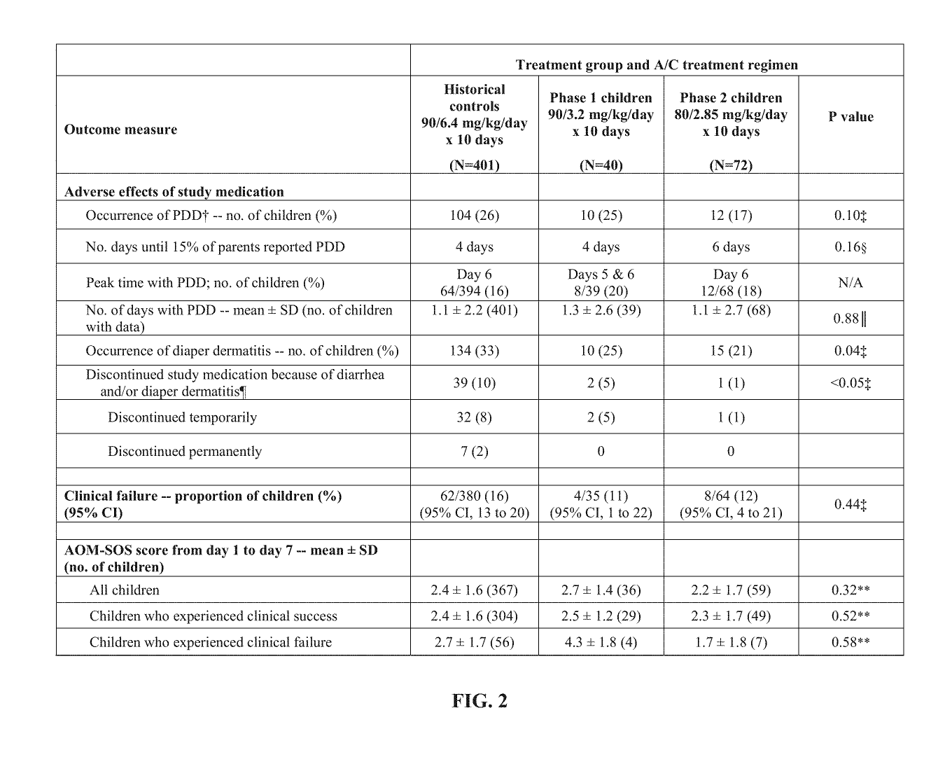

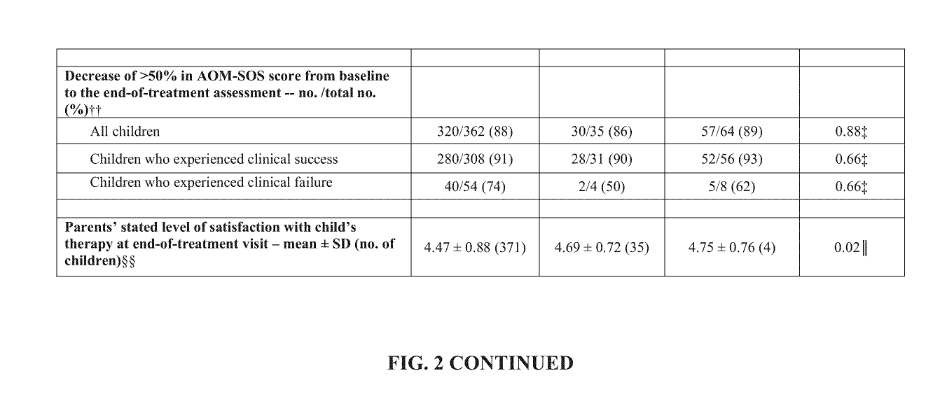

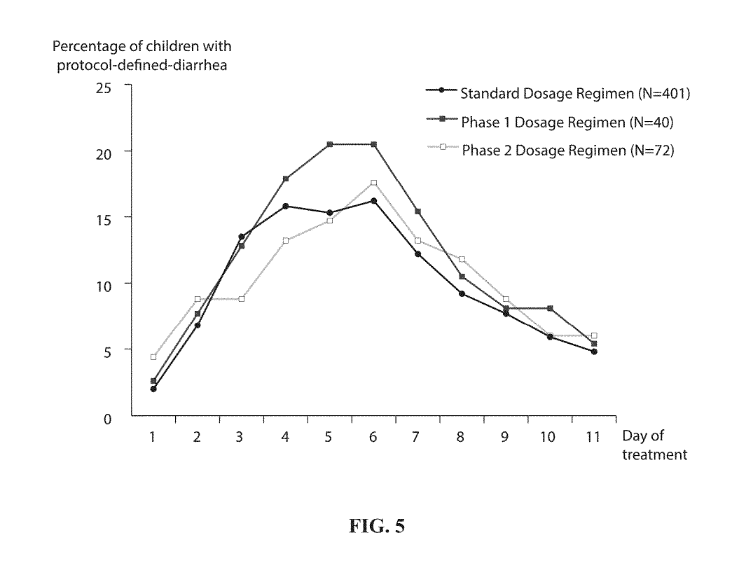

The data are summarized in FIG. 2 and FIG. 5.

Diarrhea and Diaper Dermatitis.

Compared with the proportion of children who had received the standard A/C formulation and dosing regimen (i.e. the historical controls) who experienced PDD at any time (104/401 [26%]), the proportion of Phase 1 children who did so was 10/40 (25%) (P=0.95), and of Phase 2 children was 12/72 (17%) (P=0.10). Time-to-event analysis showed no between-group differences in the number of days until 15% of parents reported PDD in their children, or in the mean number of days children experienced PDD. The proportion of children experiencing PDD peaked on or about Day 6 in each of the three study groups (FIG. 5). Compared with the proportion of historical controls who had developed diaper dermatitis that occasioned prescription of an antifungal cream, the proportion of Phase 2 children who did so was lower (134/401 [33%] vs. 15/72 [22%], P=0.04). PDD and/or diaper dermatitis resulted in discontinuation of study medication in 10%, 5% and 1% of children who received standard A/C, Phase 1 and Phase 2 formulation and dosing regimens, respectively.

Clinical Failure and Symptomatic Response.

No significant differences were observed between the historical controls, the Phase 1 children, and the Phase 2 children in the percentage categorized as experiencing clinical failure (16%, 11%, and 12%, respectively). The two-sided 95% confidence interval of the difference between the historical controls and the Phase 2 children (i.e. 4%) is expressed by (−0.06, 0.14). Key measures of symptomatic response based on parent-recorded AOM-SOS scores did not differ significantly between treatment groups. Within each treatment group, the proportion of children showing a decrease of more than 50% from baseline to the end of treatment was greater in children categorized on the basis of otoscopic findings as having experienced clinical success than in children categorized as having experienced clinical failure. At the end-of-treatment visit, parental satisfaction with their children's treatment was higher among Phase 2 children than among historical controls (on a scale of 1 to 5, mean 4.75 vs. 4.47, P=0.02).

Overall Day 7 Assessment.

At the Day 7 visit, based on AOM-attributable symptoms and otoscopic signs, study clinicians considered discontinuing antimicrobial therapy unsuitable in 11 of 34 children (32%) in Phase 1 and 31 of 62 children (50%) in Phase 2 (P=0.15). Irrespective of those judgments, parents were asked to complete the 10-day treatment course. No comparable information was available regarding historical controls.

Pharmacokinetic Characteristics.

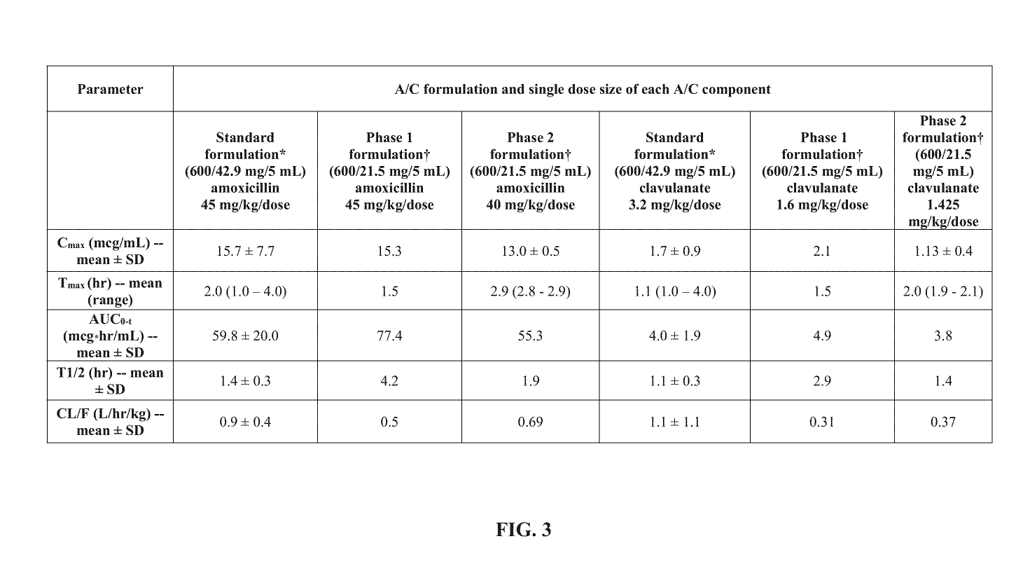

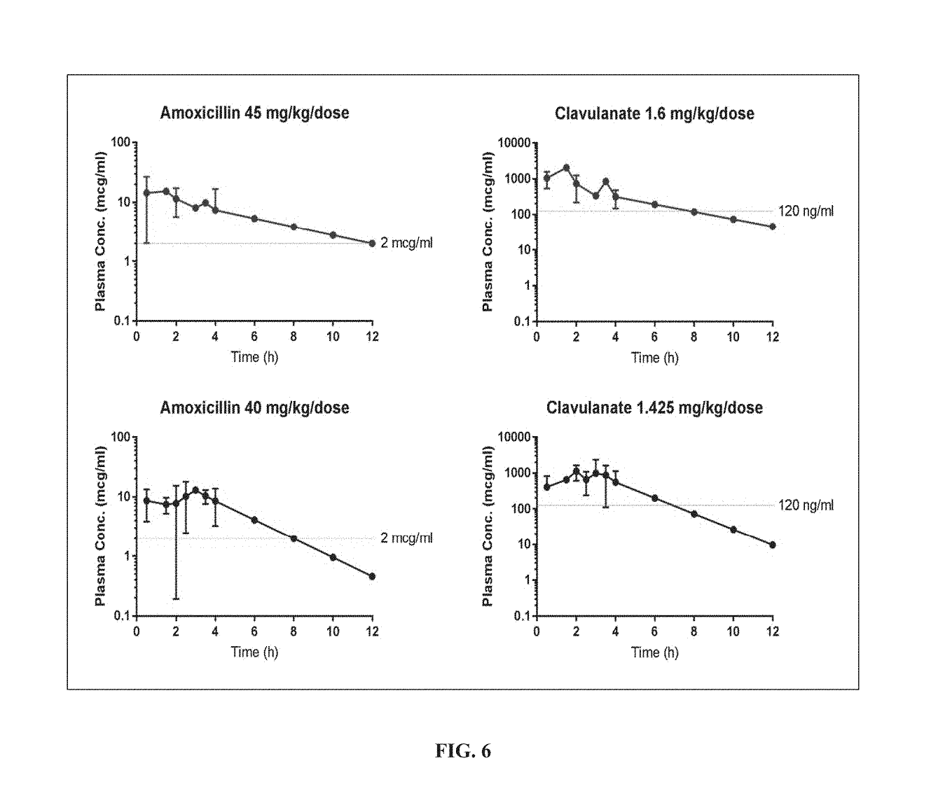

FIG. 3 shows selected pharmacokinetic findings regarding the standard, Phase 1, and Phase 2 amoxicillin-clavulanate regimens, and FIG. 6 shows population plasma concentrations of amoxicillin and of clavulanate in relation to time, for children receiving the reduced-clavulanate formulation of A/C at the respective dosing regimens of Phase 1 (90/3.2 mg/kg/day) and Phase 2 (80/2.85 mg/kg/day). At all time-points measured, the mean plasma concentration of amoxicillin was substantially higher than 1 mcg/mL and, in extrapolating the slope of the plasma level time profile beyond 4 hours, would have provided MIC levels for S. pneumoniae above 2 mcg/mL for 100% of the dosing interval in the case of the Phase 1 regimen and for 66% of the dosing interval in the case of the Phase 2 regimen—both percentages above the 40% needed to achieve optimal efficacy against S. pneumoniae.

A 55% reduction in clavulanate from the standard formulation to the Phase 2 formulation resulted in only a 33% reduction in maximum serum concentration (Cmax). The mean plasma clavulanate concentration in Phase 2 children exceeded 0.3 mcg/mL at all time points beyond 30 minutes. Both reduced-clavulanate dosing regimens resulted in plasma clavulanate levels, that would provide middle-ear fluid levels (25% to 41% as high as plasma levels), sufficient to adequately inhibit (defined as at least 50% inhibition) β-lactamases produced by H. influenzae and M. catarrhalis (0.002-0.06 mcg/mL). Further, plasma clavulanate levels greater than 0.12 mcg/mL were maintained for at least 6 hours, and would be expected, conservatively, to result in middle-ear fluid levels sufficient to inhibit 93% of β-lactamases produced by H. influenzae in an environment of 2 mcg/mL of amoxicillin.

### Discussion

In this proof-of-concept study of children aged 6 to 23 months with AOM, results of use of a novel formulation of A/C containing a reduced concentration of clavulanate, namely 21.5 mg/5 mL (providing dosages of 90/3.2 mg/kg/day or 80/2.85 mg/kg/day for 10 days), were compared against findings using the standard formulation containing 42.9 mg/5 mL (providing dosages of 90/6.4 mg/kg/day for 10 days), with the goal of determining whether use of the reduced-clavulanate formulation would result in less diarrhea and diaper dermatitis, the most common adverse side-effects of A/C use, while maintaining maximal clinical effectiveness against S. pneumoniae, H. influenzae, and M. catarrhalis. Current recommendations call for using the standard A/C formulation at a dosage of 90 mg of amoxicillin and 6.4 mg of clavulanate (90/6.4) per kg per day for 10 days. In children receiving the reduced-clavulanate formulation at a dosage of 90/3.2 mg/kg/day for 10 days (Phase 1), no appreciable differences were observed in some outcomes compared with outcomes in historical control children who had received the standard formulation and dosing regimen. Children receiving the same reduced-clavulanate formulation at a dosage of 80/2.85 mg/kg/day for 10 days (Phase 2) had a lower rate of PDD than children who had received the standard formulation, but with the difference not reaching significance (P=0.10); a significantly lower rate of diaper dermatitis (P=0.04); and a significantly lower rate of temporary or permanent discontinuation of study medication because of PDD and/or diaper dermatitis (P<0.05). There was no appreciable difference in clinical outcomes. Parental satisfaction with their children′ therapy was marginally although significantly higher among Phase 2 children than among historical controls. Satisfactory plasma levels of both amoxicillin and clavulanate were maintained in both Phase 1 and Phase 2 children.

Strengths of this study include limiting enrollment to children aged 6 to 23 months, the age group most prone to develop diarrhea and diaper dermatitis with A/C treatment; reliance on validated otoscopists who applied stringent diagnostic criteria; and use of a validated scale for rating severity of symptoms. Rates of PDD have been very consistent across the two clinical trials, which enrolled children with the same demographic and clinical characteristics, and who were diagnosed, followed, and managed by mainly the same study personnel.

Previous studies of children aged 6 to 30 months with AOM who were treated with amoxicillin (but not clavulanate) have described rates of diarrhea ranging from 10% to 17.5%, depending on the duration of treatment at the time of the report. In one of the studies, the rate in children receiving only placebo ranged from 8% to 10%. In studies over the years, differences in rates of diarrhea between children receiving varying formulations of A/C and children receiving placebo have for the most part been greater than the differences between children receiving amoxicillin alone and children receiving placebo. In two previous studies in children with AOM treated with A/C, reductions in the total daily dose of clavulanate and in the frequency of dosing resulted in reductions in the rate of diarrhea. In a review and meta-analysis of 25 randomized placebo-controlled trials involving adults and children of varying ages and with varying indications, some of whom were treated with amoxicillin alone and others with A/C, diarrhea was attributable mainly to amoxicillin-clavulanate, whereas candidiasis was attributable to both amoxicillin alone and to A/C. However, the numbers of subjects considered were small, and the reviewers concluded that underreporting of both diarrhea and candidiasis was widespread.

The disclosed novel reduced-clavulanate formulation of A/C (600/21.5 mg/5 mL) with a dosing regimen of 80/2.85 mg/kg/day for 10 days provides an improved safety profile, with rates of PDD that approximate those of amoxicillin alone, while maintaining plasma levels likely to be maximally efficacious.

### Example 4: Administration of 1.66-2.84 mg/kg/Day Clavulanate

Based on previous minimum inhibitory concentrations (MIC) data and the expectation that 25%-41% of blood levels are present in middle-ear fluid, it was predicted that the clavulanate dose can be reduced from 6.4 mg/kg/day to 3.2 mg/kg/day (50% reduction) administered in two divided doses. Based on previous pharmacokinetic/pharmacodynamics (PK/PD) clavulanate data, a linear decrease in clavulanate could be assumed. The maximum concentration (Cmax) was therefore calculated to be reduced by 55% (1.7 μg/mL to 0.77 μg/mL) compared to the standard formulation when a final dose of 2.85 mg/kg/day was used.

Actual results showed a Cmax reduction of only 33% (1.7 μg/mL to 1.13 μg/mL). This likely resulted from increased clavulanate absorption or reduced clavulanate clearance, or both, due to a less developed gastrointestinal tract or kidneys, respectively, in children less than 2 years of age as compared to older children. This Cmax finding represents a 48% increase over the calculated expectation if linearity were observed (0.765 μg/mL×1.48=1.13 μg/mL).

Accordingly, the Cmax observed in the PK/PD data provided justification to further lower the minimal clavulanate dose to 1.66 mg/kg/day (3.2 mg/kg/day×0.52, or 48% lower).

Further, the area under the concentration curve (AUC) observed in the PK/PD study in children less than 2 years of age was only 5% less than that previously reported in older children for the standard formulation (4 g h/mL for 6.4 mg/kg/day to 3.8 h/mL for 2.85 mg/kg/day, a resultant 5% reduction). Based on this observed AUC, use of a final dose of 2.85 mg/kg/day+5% provides the same clavulanate AUC as the standard dosing of 6.4 mg/kg/day. The surprising and unexpected PK/PD results in children under age 2 which showed increased gastrointestinal absorption or decreased kidney clearance of clavulanate, or both, provided justification to adjust the maximum clavulanate dose to 2.99 mg/kg/day (2.85 mg/kg/day+5%) to replicate the efficacy of standard clavulanate dosage.

The observed effective treatment having lower but not significantly reduced rate of PDD in children receiving 3.2 mg/kg/day clavulanate compared to those receiving the standard formulation justified further reducing the maximum clavulanate dosages to 2.85 mg/kg/day. It was a surprising finding that very small amounts of clavulanate dosages, for example from 1.66 mg/kg/day to 2.99 mg/kg/day, can reduce side effects while maintaining therapeutic effectiveness. Thus, dosages of 1.66-2.99 mg/kg/day, desirably 1.66-2.84 mg/kg/day or 1.66-2.5 mg/kg/day, are administered in two divided doses as a surprisingly effective therapy against acute otitis media and other bacterial infections while minimizing adverse side effects such as PPD.

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

What is claimed is:

1. A method of treating pediatric otitis media in a patient under 24 months of age, the method comprising administering amoxicillin and clavulanate potassium to the patient wherein an amoxicillin dosage is at least about 40 mg/kg/day and a clavulanate potassium dosage is from about 1.66 mg/kg/day to about 2.84 mg/kg/day.

2. The method of claim 1, wherein the amoxicillin dosage is in a range from about 80 mg/kg/day to about 90 mg/kg/day.

3. The method of claim 1, wherein the clavulanate potassium dosage is in a range from about 1.66 mg/kg/day to about 2.5 mg/kg/day.

4. The method of claim 1, wherein the clavulanate potassium dosage is in a range from about 1.66 mg/kg/day to about 2.0 mg/kg/day.

5. The method of claim 1, wherein the amoxicillin and the clavulanate potassium are administered in a molecular weight ratio ranging from about 15:1 to about 56:1.

6. The method of claim 1, wherein the method reduces a rate of protocol-defined diarrhea (PDD) by at least 9 percent compared to a control.

7. The method of claim 1, wherein the method reduces a rate of diaper dermatitis by at least 8 percent compared to a control.

8. The method of claim 1, wherein the patient has had frequent exposure to other children and/or has been previously treated with one or more antibiotics.

9. The method of claim 1, wherein a first daily administration comprises more clavulanate potassium than a second daily administration.

10. The method of claim 1, wherein the dosages are administered daily for fourteen days or less.

11. The method of claim 1, further comprising administering a pain reduction medication.

12. The method of claim 11, wherein the pain reduction medication is selected from acetaminophen, non-steroidal anti-inflammatory medication, and antipyretic medication.

13. A method of treating a pediatric patient under 24 months of age for a drug resistant bacterial infection, the method comprising administering amoxicillin and clavulanate potassium to the patient wherein an amoxicillin dosage is at least about 40 mg/kg/day and a clavulanate potassium dosage is from about 1.66 mg/kg/day to about 2.84 mg/kg/day.

14. The method of claim 13, wherein the amoxicillin dosage is in a range from about 80 mg/kg/day to about 90 mg/kg/day.

15. The method of claim 13, wherein the clavulanate potassium dosage is in a range from about 1.66 mg/kg/day to about 2.5 mg/kg/day.

16. The method of claim 13, wherein the amoxicillin and the clavulanate potassium are administered in a molecular weight ratio ranging from about 28:1 to about 56:1.

17. The method of claim 13, wherein the antimicrobial resistant bacterial infection is selected from Streptococcus pneumoniae, Haemophilus influenzae, and Moraxella catarrhalis.

18. A method of treating a beta-lactamase producing Haemophilus influenzae or Moraxella catarrhalis in a pediatric patient under 24 months of age, the method comprising administering amoxicillin and clavulanate potassium to the patient wherein an amoxicillin dosage is at least about 40 mg/kg/day and a clavulanate potassium dosage is from about 1.66 mg/kg/day to about 2.84 mg/kg/day.

19. The method of claim 18, wherein the amoxicillin dosage is in a range from about 80 mg/kg/day to about 90 mg/kg/day.

20. The method of claim 18, wherein the clavulanate potassium dosage is in a range from about 1.66 mg/kg/day to about 2.5 mg/kg/day.

21. The method of claim 18, wherein the amoxicillin and the clavulanate potassium are administered in a molecular weight ratio ranging from about 28:1 to about 56:1.

22. A method of treating pediatric otitis media in a patient under 24 months of age, the method comprising administering amoxicillin and clavulanate potassium to the patient wherein an amoxicillin dosage is from about 40 mg/kg/day to about 50 mg/kg/day, and a clavulanate potassium dosage is from about 2.85 mg/kg/day to about 3.2 mg/kg/day.
